(19) United States
(12) Patent Application Publication
(10) Pub. No.: US 2010/0125045 A1
(43) Pub. Date: May 20, 2010

(54) COSMETIC OR TOILET PREPARATION

(76) Inventors: Iryna URSIC, Sempeter Pri Gorici (SI); Anna TSYBULSKA, Lviv (UA)

Correspondence Address: BEEM PATENT LAW FIRM, 53 W. JACKSON BLVD., SUITE 1352, CHICAGO, IL, 60604-3787, US

(21) Appl. No.: 12/619,943

(22) Filed: Nov. 17, 2009

(30) Foreign Application Priority Data
Nov. 18, 2008 (SI) SI P-200800284

(51) Int. Cl. A61K 8/02; A61Q 19/10

(52) U.S. Cl. 510/147; 510/141

(57) ABSTRACT

The present invention relates to cosmetic or similar toilet preparations in particular to a container for storing and/or use of a cosmetic or similar toilet preparation in solid form, and preferably to a container for storing and/or use of a soap for personal use. It is provided for, according to the invention, that a container (1) which can be separated along a dividing line (4), comprises a layer (6) of a cosmetic or toilet preparation being applied onto said container (1).

### CROSS-REFERENCE TO RELATED APPLICATIONS

This application claims the benefit of priority from Slovenian Patent Application P-200800284, filed Nov. 18, 2008.

### BACKGROUND OF THE INVENTION

1. Field of the Invention

The present invention relates to cosmetic or similar toilet preparations in particular to a container for storing and/or use of a cosmetic or similar toilet preparation in a solid form, and preferably to a container for storing and/or use of a soap for personal use.

2. Description of the Related Art

It has been found out that in public spaces where abundance of people using soap exchange every day, such as in hotels and similar facilities, the users are still provided with the soap in a solid form such as a massive block having an arbitrary outside form. However, the soap consumption in a solid form is relatively high and inappropriate with regard to the recent environmental requirements since the users discard the soap before they use it entirely, because the users cannot hold the soap in their hands due to the wear and consequent volume reduction thereof up to the entire use of the soap.

### SUMMARY OF THE INVENTION

In one aspect of the invention, a cosmetic or toilet preparation is provided. The cosmetic or toilet preparation includes a container (1) which can be separated along a dividing line (4), and a layer (6) of a cosmetic or toilet preparation being applied onto said container (1).

### BRIEF DESCRIPTION OF THE DRAWINGS

FIG. 1 shows an embodiment of the invention.

### DETAILED DESCRIPTION OF EMBODIMENTS OF THE INVENTION

It is the object of the present invention to create a cosmetic or similar toilet preparation, in particular for personal use, remedying therewith drawbacks of the existing soap.

According to the invention, the object as set above is solved by a container which can be separated along a dividing line, said container being covered with a layer of a cosmetic or a toilet preparation, preferably said cosmetic or toilet preparation being applied onto said container. Here, said container consists of two halves that may be separated and/or joined in a snap like manner along said dividing line extending over at least a part of the periphery of said container. It is provided for, according to the invention, that at least one half of said container is made of a translucent material, preferably of a transparent material, thus enabling a view inside the said container where, optionally, an object is placed such as a toy or similar. Furthermore, it is provided for according to the invention that said layer of the cosmetic or toilet preparation comprises a relatively small thickness.

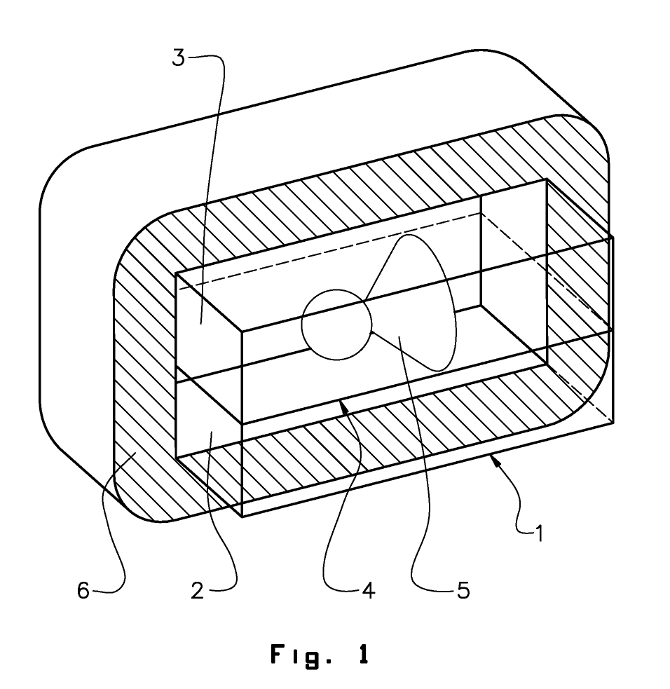

The invention will be more readily understood on reading the following description with reference to the accompanying drawing, FIG. 1, showing a cross-section of a three-dimensional view of a soap according to the invention.

The present invention consists of a container 1 being preferably formed in a manner that it can be divided along a dividing line 4, so that both halves 2, 3 can be mutually separated and/or joined in a manner known per se, by means of snapping of said halves 2, 3 for instance. According to the invention it is provided for that said dividing line 4 extends at least over a part of the periphery of said container 1 and preferably, it extends over the entire periphery of said container 1. Optionally, an object 5 such as a toy or similar is arranged inside said container 1. Furthermore, it is provided for according to the invention that said container 1 is covered with a layer 6 of a cosmetic or toilet preparation, a soap for example. Preferably, said layer 6 is applied onto said container 1 in a manner that said layer 6 cannot detach itself from the container 1 but can be removed only by the regular use of the soap.

It is provided for according to the invention that at least one of said halves 2, 3 of said container 1 is made of a transparent material, thus enabling the user to see inside said container 1. In this way, the user, such as a child for example, who uses the subject of the invention, such as a soap for example, is encouraged to use said soap, since the curiosity drives the child to see what object is hidden inside said container 1. It is provided for according to the invention that said layer 6 of said cosmetic or toilet preparation comprises a relatively small thickness resulting in faster use of said cosmetic or toilet preparation and the user may faster reach said container 1 and the content 5 thereof.

We claim:

1. A cosmetic or toilet preparation comprising:
a container (1) which can be separated along a dividing line (4), and
a layer (6) of a cosmetic or toilet preparation being applied onto said container (1).

2. A cosmetic or toilet preparation according to claim 1, wherein said container (1) consists of two halves (2, 3) that may be separated and/or joined along said dividing line (4) extending over at least a part of the periphery of said container (1), preferably extending over the entire periphery of said container (1).

3. A cosmetic or toilet preparation according to claim 1, wherein said halves (2, 3) may be separated and/or joined in a snap like manner along said dividing line (4).

4. A cosmetic or toilet preparation according to claim 1, wherein at least one half (2, 3) of said container (1) is made of a translucent material, preferably a transparent material.

5. A cosmetic or toilet preparation according to claim 1, further comprising an object (5), such as a toy or similar, arranged within said container (1)

6. A cosmetic or toilet preparation according to claim 1, wherein said layer (6) of the cosmetic or toilet preparation being applied onto the container (1) comprises a relatively small thickness.
